(19) United States
(12) Patent Application Publication
(10) Pub. No.: US 2022/0217998 A1
(43) Pub. Date: Jul. 14, 2022

(54) FEED COMPOSITIONS CONTAINING BETAINE SALTS

(71) Applicant: Evonik Operations GmbH, Essen (DE)

(72) Inventors: Ursula RIESEN, Alzenau (DE); Frank Wilz, Alzenau (DE); Lorena Stannek-Göbel, Bielefeld (DE); Adriana Barri, Goecklingen (DE); Stefan Pelzer, Guetersloh (DE); Ulrike Kottke, Linsengericht-Grossenhausen (DE); Christoph Kobler, Alzenau (DE); Achim Fischer, Goldbach (DE); Vincent Hess, Hanau (DE)

(73) Assignee: Evonik Operations GmbH, Essen (DE)

(21) Appl. No.: 17/608,274

(22) PCT Filed: Apr. 28, 2020
(86) PCT No.: PCT/EP2020/061700

§ 371 Date: Nov. 2, 2021

(30) Foreign Application Priority Data
May 3, 2019 (EP) 19172517.5

(51) Int. Cl. A23K 10/18; A23K 20/105; A23K 20/22; A23K 40/10; A23K 50/75; A61K 35/742; A61K 35/744; A61K 9/00

(52) U.S. Cl. CPC A23K 10/18; A23K 20/105; A23K 20/22; A61K 9/0056; A23K 50/75; A61K 35/742; A61K 35/744; A23K 40/10

(57) ABSTRACT

Compositions, in particular compacted compositions, contain at least one betaine salt and at least one feed additive; and can be used for providing the feed additive, in particular probiotics, to drinking water or rearing water.

The invention relates to compositions, in particular compacted compositions, containing at least one betaine salt and at least one feed additive and their use for providing the feed additive, in particular probiotics, to drinking water or rearing water.

Probiotics (also known as direct-fed microbials) are nowadays generally recognized as suitable feed additives. Primarily they serve as substitutes for antibiotics. Besides their use as feed ingredients, it has been already disclosed further that the probiotics alternatively might be applied to drinking water. In this context, different application forms of probiotics have been disclosed in the state of the art.

Thus, for example WO 2008/107746 discloses a tablet containing probiotic microorganisms and high amounts of polysaccharides (40-80 wt.-% of microcrystalline cellulose and 8-25 wt.-% of sodium carboxymethyl cellulose) as disintegration aid.

According to the invention, it was surprisingly found out that feed compositions, in particular compacted feed compositions, do dissolve fast without leaving unwanted remains and further effect a homogeneous distribution of the feed additive, in particular probiotic, if they contain a substantial amount of a betaine salt.

Further it was found out that the formation of an undesirable biofilm in the water reservoir can be avoided by making use of the compositions of the present invention.

In addition the compacted compositions of the invention allow a comfortable handling and an easy application of the feed additive to the animals. Furthermore they exhibit very good storage stability.

Thus, a first subject matter of the present invention is a composition, comprising at least one betaine salt and at least one feed ingredient.

Betaine is also known as trimethylglycine or 2-trimethylammonioacetate. Principally, all kinds of betaine salts can be used according to the invention. Salts of betaine which are preferably used according to the present invention are salts of betaine with strong acids, in particular with strong inorganic acids, with betaine hydrochloride being particularly preferred. Examples of further betaine salts are in particular betaine bromide and betaine salicylate.

Preferably the betaine salt, in particular betaine hydrochloride, is contained in the composition in an amount of 20 to 90 wt.-%, in particular 30 to 80 wt.-%, more preferably 40 to 75 wt.-%, above all in an amount of 45 to 65 wt.-%. Independent of the fact whether the composition comprises only one betaine salt or a mixture of different betaine salts, the preferred ranges as depicted before relate also to the total amount of betaine salts as contained in the composition.

The composition according to the invention can for example be a powder, granules or a gel, but is preferably a compacted composition, in particular a compacted powder or compacted granules. Thus, a preferred subject matter of the present invention is in particular a compacted composition, which contains at least one betaine salt, in particular betaine hydrochloride, in an amount of 20 to 90 wt.-%, preferably in an amount of 30 to 80 wt.-%, more preferably in an amount of 40 to 75 wt.-%, above all in an amount of 45 to 65 wt.-%, wherein independent of the fact whether the composition comprises only one betaine salt or a mixture of different betaine salts, the preferred ranges as depicted before relate also to the total amount of betaine salts as contained in the composition.

“Compacted (feed) composition” according to the invention in general refers to a composition, containing at least one betaine salt and at least one feed additive, wherein compacting was carried out by applying external pressure. The compacted feed composition according to the invention is preferably a tablet.

A further subject matter of the present invention is therefore also a method of preparing a compacted composition according to the present invention, comprising the steps of providing a granular composition comprising all ingredients of the composition, in particular at least one betaine salt and at least one feed additive, and subsequently pressing the granular composition, preferably after adding a binder, to form the compacted composition, in particular a tablet.

The granular composition which is used for preparing the compacted composition has preferably a water content of less than 3 wt.-%, in particular of 0.5 to 3 wt.-%. It further preferably comprises particles with an average particle size of 2 μm to 6 mm, in particular of 5 μm to 5 mm. The granular composition is preferably obtained by first providing the different components of the composition in powdery form and subsequently carrying out a granulation of these components, for example by fluidized bed granulation.

Compacting of the composition is preferably carried out by applying a pressure force of 5 to 100 kN, preferably 10 to 50 kN, in particular 20 to 40 kN, Compacting is preferably carried out by using a tablet press. As press for carrying out the compaction, for example a rotary press or an eccentric press can be used.

The compacted feed composition, in particular the tablet, exhibits preferably a hardness of 80 to 150 N, in particular 90 to 130 N. The hardness of the composition, independent of its shape, can for example easily be determined by using a MultiTest 50 Tablet Hardness Tester (Dr. Schleuninger® Pharmatron, Sotax AG, Switzerland).

The composition can have any kind of spatial shape, in particular it can have the shape of a sphere, a cube, a cone, a cylinder, a prism, a pyramid, a tetrahedron or a tablet. Preferably it has the shape of a tablet with two equidistant planes, in particular with circular or angular shape of the planes, wherein tablets with circular shape of the planes are very preferred

The compacted feed composition, in particular the tablet, has preferably a diameter of 10 to 50 mm, in particular 20 to 40 mm, more preferably 25 to 35 mm. The tablet further preferably has a thickness of 25 to 140 mm, more preferably of 60 to 100 mm.

The compacted feed composition, in particular the tablet, has further preferably a weight of 3 to 25 g, in particular 6 to 20 g, more preferably 6 to 15 g. It further has preferably a volume of 3 to 15 ml, more preferably 4.5 to 9 ml.

The betaine salt is functioning in the composition as proton donor and by that enables a fizzy behavior in the presence of carbonate and/or bicarbonate salts, when dissolved in water. Thus, to enable the fizzy behavior, the compositions according to the present invention preferably also comprise at least one carbonate and/or bicarbonate salt.

The carbonate salts are, if present, preferably present in the compositions according to the invention in an amount of 10 to 35 wt.-%, in particular in an amount of 15 to 30 wt.-%, more preferably in an amount of 20 to 30 wt.-%. Principally, all kinds of carbonate salts can be used according to the invention. Preferred carbonate salts according to the invention are salts of carbonic acid with strong bases, in particular with strong inorganic bases. A very preferred carbonate salt as used according to the invention is sodium carbonate. The preferred ranges as depicted before relate to the total amount of carbonate salts as contained in the composition, independent of the fact, whether the composition contains only one, two or several different carbonate salts.

Besides or instead of carbonate salts preferably also at least one bicarbonate salt is present in the composition. If present, bicarbonate salts are preferably present in the composition in an amount of 1 to 10 wt.-%, more preferably in an amount of 2 to 8 wt.-%, in particular in an amount of 3 to 6 wt.-%. Principally, all kinds of bicarbonate salts can be used according to the invention. Preferred bicarbonate salts according to the invention are salts of protonated carbonic acid with strong bases, in particular with strong inorganic bases. A very preferred bicarbonate salt as used according to the invention is sodium bicarbonate. The preferred ranges as depicted before relate to the total amount of bicarbonate salts as contained in the composition, independent of the fact, whether the composition contains only one, two or several different bicarbonate salts.

In addition to the betaine salt, the composition may comprise further proton donors in smaller amounts, in particular selected from polyfunctional acids, preferably selected from polyfunctional fruit acids, in particular selected from citric acid, adipic acid, succinic acid, glutaric acid, malic acid, tartaric acid, gluconic acid, and salts of those acids, and mixtures of said compounds. If present, the further proton donors are preferably present in a total amount of less than 20 wt.-%, more preferably in an amount of 0.1 to 15 wt.-%, in particular in an amount of 5 to 15 wt.-%.

In a preferred embodiment of the invention, the composition comprises citric acid or a salt thereof in an amount of 2 to 20 wt.-%, in particular in an amount of 5 to 15 wt.-%.

As the use of polyfunctional acids, in particular citric acid, has a detrimental effect on the dissolution time of the compacted composition and also impairs the release of the compacted composition from the matrix of the pressing machine, the use of higher amounts of polyfunctional acids, in particular citric acid, is preferably avoided.

In addition, the compositions, in particular the compacted compositions, may also comprise further disintegrants like cellulose and derivatives, in particular crystalline cellulose and methyl cellulose, starch, in particular potatoe or corn starch, alginic acid, salts of alginic acid or casein, or modified forms of those compounds, or combinations thereof. If present, those further disintegrants are preferably present in a total amount of less than 5 wt.-%, in particular in an amount of 0.1 to 5 wt.-%, more preferably in an amount of 0.1 to 3 wt.-%.

The composition may further contain substances suitable for stabilizing the composition and/or for improving the properties of the composition and/or facilitating the preparation or dissolution of the composition. These components may in particular be selected from flow regulators, anti-caking agents, lubricants, emulsifiers, antioxidants, bulking agents, gelatinizers, colouring agents and flavouring agents.

Examples for flow regulators or anti-caking agents which can be used according to the invention are silica, silicates, in particular aluminium, magnesium and/or calcium silicate, clays, zeolites,

Fuller's earth, montmorillonite, diatomaceous earth, talc, bentonites, trisodium citrate, glycerol or 1,2-propanediol. In a preferred embodiment the composition comprises flow regulators and/or anti-caking agents, preferably silica, in a total amount of 0.2 to 5 wt.-%, in particular in a total amount of 0.5 to 3 wt.-%. The silica may in particular be selected from hydrophobic, hydrophilic, amorphous, fumed and/or precipitated silica. Silicas preferably used according to the invention are obtainable under the trademark Sipernat® (Evonik Industries, Germany).

If lubricants are used, they are preferably used in an amount of 0.1 to 10 wt.-%, in particular in an amount of 0.1 to 5 wt.-%, and are preferably selected from talcum, isoleucine, magnesium stearate, glycerol monostearate, monolaurate and monopalmitate esters of sugar molecules, in particular sucrose monolaurate and sucrose monopalmitate, adipic acid and their salts, fumaric acid and their salts, benzoic acid and their salts, glycerol triacetate, sodium and magnesium lauryl sulfate, polyethylene glycols, in particular PEG 4000 to PEG 6000, and esters thereof with fatty acids, polyvinyl pyrrolidone, skim milk powder, leucine, glycine, boric acid, sodium propionate, salts of fatty acids, sodium acetate, hydrogenated vegetable oils, and mixtures of those compounds. In a very preferred embodiment of the invention the lubricant comprises a mixture of fumaric acid, sucrose stearate and PEG 6000 stearate.

Lubricants are in particular used to facilitate the detachment of the compacted composition from the matrix of the pressing machine. The lubricant can be contained in the composition itself or alternatively put on the composition and/or to the matrix right before the compacting step.

If emulsifiers are used, then they are preferably also used in small amounts, in particular in a total amount of 0.1 to 3 wt.-%. An example for a preferred emulsifier is lecithin. Emulsifiers stabilize the mixture of the components as contained in the composition.

Examples for suitable anti-oxidants which can be used according to the invention are gallates, L-ascorbic acid, lycopene, beta-carotene, lutein, hydroxytyrosole, tocopherols, butylated hydroxyanisole (BHA), butylhydroxytoluene, homocysteine, cystathionine, glutathione, salts thereof and combinations thereof and peptides containing homocysteine, cysteine, cystathionine, methionine and/or gluathione, and combinations thereof. Anti-oxidants are, if used, preferably also present only in small amounts, in particular in a total amount of 0.1 to 3 wt.-%.

Examples for gelatinizers which can be used according to the invention are: alginic acid, sodium alginate, potassium alginate, ammonium alginate, calcium alginate, agar, carrageenan, locust bean gum, guar gum, guar flour, tragacanth, konjac glucomannan, propylene glycol alginate, PES, acacia gum, xanthan gum, karaya gum, tara gum, gellan gum and combinations thereof. Gelatinziers are, if used, preferably also present only in small amounts, in particular in a total amount of 0.1 to 3 wt.-%.

The compositions according to the invention may also comprise further inert formulation additives like maltodextrins, chitosan and/or inulins, yeast extract, caseinate, sodium starch glycolate, stearic acid, polyols, in particular sugar alcohols (in particular mannitol), protein sources (in particular sweet-whey powder), peptides, sugars (in particular lactose, trehalose, sucrose, saccharides and/or dextrose), lipids (in particular vegetable oils and/or mineral oils), salts (in particular sodium chloride, sodium carbonate, calcium carbonate, chalk, limestone, magnesium carbonate, sodium phosphate, calcium phosphate, in particular anhydrous dicalcium phosphate, magnesium phosphate), and combinations thereof. Suitable inert feed additives are set forth in the American Feed Control Officials, Inc.' s Official Publication, which publishes annually. See, for example Official Publication of American Feed Control Officials, Sharon Krebs, editor, 2006 edition, ISBN 1-878341-18-9.

In a preferred embodiment of the invention, the compositions according to the invention comprise calcium phosphate, in particular anhydrous dicalcium phosphate, in an amount of 0.1 to 10 wt.-%, preferably in an amount of 0.2 to 3 wt.-%.

Preferably, the compositions according to the invention due to their ingredients already show only a very low inclination to absorb humidity, i.e. they are very stable over time, without exhibiting unwanted caking.

For further protection against humidity, the compositions, in particular the compacted compositions according to the invention may be stored in a sheath, in particular in a water-soluble sheath.

Alternatively they may also be stored and marketed in tubes or blister packs.

In a preferred embodiment of the invention, the compositions are free of glucose, preferably free of any monosaccharides and more preferably also free of any disaccharides and above all free of any sugar molecules, including in particular mono-, oligo- and polysaccharides, at all, or contain glucose, preferably monosaccharides in general, more preferably also disaccharides in general, above all any sugar molecules, only in very small amounts, preferably in an amount of not more than 1 wt.-%, in particular in an amount of less than 0.5, 0.2 or 0.1 wt.-%. This is in particular the case, because the presence of sugar molecules, in particular low molecular sugar molecules, may promote the growth of microorganisms in the water reservoir and by that may promote the formation of an undesired biofilm.

Further the compositions according to the present invention are preferably also free of CaCO₃ or contain CaCO₃, if present, only in very small amounts, preferably in an amount of not more than 0.5 wt.-%, in particular in an amount of less than 0.2 or 0.1 wt.-%. This is in particular the case, because the presence of CaCO₃ in the compositions may lead to the formation of undesired precipitates, which in particular may lead to a blockage of the drinking nipples.

Before application of the compositions to the animals, the compositions are preferably dissolved in water or in an aqueous solution, in particular in drinking water or rearing water. The compositions as thus obtained by dissolving the compositions are also called “(aqueous) solutions” or “(aqueous) solutions according to the (present) invention” in the following.

The composition according to the invention preferably exhibits a pH value of 4.5 to 7.5, in particular 5 to 7, more preferably 5.5 to 6.5, when dissolved in distilled water (5 wt.-% of the composition dissolved in distilled water at 20° C.).

The dissolution speed of the compositions, in particular the compacted compositions, is preferably less than 15 minutes, more preferably less than 10 minutes.

According to the invention, one composition according to the invention, in particular one compacted composition, in particular one tablet, is preferably dissolved in a volume of 800 to 1200 liter of water, preferably in a water tank, water pond or water reservoir. To accelerate the dissolution of the tablet and/or the homogeneous distribution of the feed additives in the water, the water may be mixed after addition of the tablet, for example by stirring.

Alternatively, a more concentrated stock solution can be prepared in a first step by first dissolving the composition, in particular tablet, in a smaller water volume, for example by dissolving the composition in 20 to 200 liter of water as a first step, and this stock solution is then used for preparing the more diluted solutions which are provided to the animals.

The feed additives as contained in the compositions according to the invention are preferably probiotics or a preparation thereof. Thus, a preferred subject matter of the present invention is a composition, in particular compacted composition, comprising at least one betaine salt and at least one probiotic.

But besides probiotics or instead of probiotics other feed additives can be contained in the compositions as well. These other feed additives are in particular selected from minerals, vitamins, enzymes, prebiotics, amino acids, organic acids, vaccines and immune modulators.

Thus, the feed additives as contained in the composition can in particular be selected from probiotics, minerals, vitamins, enzymes, prebiotics, amino acids, organic acids, vaccines, immune modulators and any combinations thereof.

The feed additives, in particular the probiotics, are according to the invention preferably contained in the composition in an amount of up to 25 wt.-%, in particular in an amount of 1 to 25 or 1 to 20 wt.-%, more preferably in an amount of 2 to 18 wt.-%, above all in an amount of 5 to 15 wt.-%.

The probiotics according to the invention are preferably probiotic microorganisms, in particular probiotic bacteria, and preferably selected from Bacillus, in particular B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, Enterococcus, in particular E. faecium and E. faecalis, Geobacillus, in particular G. stearothermophilus, Clostridium, in particular C. butyricum, and Streptococcus, in particular S. faecalis, S. faecium, S. gallolyticus, S. salivarius subsp. thermophilus and S. bovis, Lactobacillus, in particular L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius and L. sanfranciscensis, Pediococcus, in particular P. acidilactici, P. dextrinicus and P. pentosaceus, Streptococcus, in particular S. lactis and S. thermophiles, Bifidibacterium, in particular S. adolescentis, B. animalis, B. bifidum, B. breve and B. longum, or any combinations thereof. In a very preferred embodiment of the invention the probiotic microorganisms are of the genus Bacillus, in particular selected from the following strains and combinations thereof: B. subtilis DSM 32315, B. subtilis DSM 32540, B. subtilis DSM 32592, B. licheniformis DSM 32314, B. pumilus DSM 32539, B. amyloliquefaciens CECT 5940.

Thus, a very preferred subject matter of the present invention is a composition, in particular compacted composition, comprising at least one betaine salt and at least one probiotic of the genus Bacillus, in particular selected of the species B. subtilis, B. licheniformis, B. pumilus, B. amyloliquefaciens and mixtures thereof.

In a further very preferred embodiment of the invention the probiotic microorganisms are of the genus Enterococcus, with the strain Enterococcus faecium CECT 4515 being preferred.

The probiotics may be present in the compositions of the present invention as spores (which are dormant), as vegetative cells (which are growing), as transition state cells (which are transitioning from growth phase to sporulation phase) as well as in an inactivated state, or as a combination of at least two or three, in particular all of these types of cells. In a preferred embodiment, the composition of the present invention comprises mainly or only spores.

The probiotic microorganisms which are used according to the invention are preferably used in the form of particles with an average particle size (d50) of 0.2 to 5 μm, more preferably 0.4 to 3 μm, above all 0.6 to 1.2 μm. Particles with that particle size can be obtained for example by spray-drying. The small particle size facilitates the homogeneous distribution of the probiotics in the water reservoir and prevents sedimentation of the probiotics.

The probiotic microorganisms as used according to the invention may be provided in a carrier-bound form as well as free of any carriers. Suitable carriers for probiotics are known to those skilled in the art and are for example disclosed in WO 2017/207372.

In case that probiotics are used as feed additives, the spore count in the composition is preferably from 1×10¹⁰to 1×10¹³, preferably from 1×10¹¹ to 1×10¹² CFU/g.

Preferred compositions of the present invention, in particular those which contain at least one probiotic, when administered to animals after dissolution in water preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce the pathogen shedding through the feces of such animals. In particular compositions of the present invention might be used after dissolution in water to assist in re-establishing a healthy balance of the gut microflora after administration of antibiotics for therapeutic purposes.

A further subject of the present invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or of reducing the pathogen shedding through the feces of animals, wherein such preferred compositions of the present invention, in particular those which contain at least one probiotic, are dissolved in water and subsequently administered to animals.

“Increasing the productivity of animals” refers in particular to any of the following: production of more or higher quality eggs, milk or meat or increased production of weaned offspring.

Preferred compositions according to the invention, in particular those which contain at least one probiotic, can in particular also be used for improving the quality of water. A further subject of the present invention is therefore also a method of controlling and/or improving the quality of water or aqueous solutions, in particular of drinking water and/or rearing water, comprising the step of applying to water such a preferred composition according to the present invention.

Further, the compositions according to the invention can also be used for treating plants, in particular for treating microbial diseases of plants. A further subject of the present invention is therefore also a method of treating plants, in particular a method of treating and/or preventing microbial diseases of plants, in particular of cultivated plants, comprising the step of applying to the plants a composition of the present invention. The application may be carried out for example by first dissolving the composition in a reservoir and subsequently applying the solution such obtained in liquid form, for example by spraying.

In particular, the compositions of the present invention, in particular those containing at least one probiotic, may also be administered to an animal after dissolution in water in an amount effective to inhibit and/or decrease the growth and/or prevent the outgrowth of pathogenic bacteria in the animal gut. Such pathogenic bacteria include Clostridia, Listeria, Salmonella, Enterococci, Staphylococci, Aeromonas, Streptococci, Campylobacter, Escherichia coli, and Vibrio. Relatedly, the methods of the present invention may be used to decrease the amount of pathogenic bacteria shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora.

Thus, a further subject of the present invention is a method of inhibiting and/or decreasing the growth and/or preventing the outgrowth of harmful or pathogenic bacteria and/or maintaining and/or increasing the growth of beneficial bacteria in an animal gut, wherein a composition of the present invention, in particular a composition which contains at least one probiotic, is dissolved in water and subsequently administered to animals and wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gallinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gallinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

In a preferred embodiment of the invention the amount of at least one pathogenic bacterium, in particular the amount of C. perfringens, is reduced by at least 0.5 log, more preferably by at least 1 log, 2 log, or 3 log.

Thus, a further subject of the present invention are also compositions of the present invention, in particular those containing at least one probiotic, for inhibiting and/or decreasing the growth and/or preventing the outgrowth of pathogenic bacteria and/or for maintaining and/or increasing the growth of beneficial bacteria in an animal gut, wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gaffinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gaffinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

The occurrence and/or increased growth of the pathogenic bacteria does or can lead to the outbreak of certain diseases. For example, the occurrence and/or increased growth of Clostridium perfringens can lead to the outbreak of gut diseases, in particular to the outbreak of necrotic enteritis in poultry. The occurrence and/or increased growth of Clostridium perfringens can also lead to the outbreak of further diseases like bacterial enteritis, gangrenous dermatitis and colangiohepatitis. Even the mildest form of infection by C. perfringens can already be accompanied by diarrhea, which results in wet litter and by that may lead to secondary diseases like foot pad dermatitis.

A further subject of the present invention is therefore also the use of the compositions according to the present invention as therapeutic compositions.

A preferred subject in this context is therefore a therapeutic composition for treatment and/or prevention of necrotic enteritis, in particular sub-clinical necrotic enteritis, in animals, preferably poultry, wherein the therapeutic composition is a compacted composition according to the present invention.

Another preferred subject in this context is therefore a therapeutic composition for treatment and/or prevention of bacterial enteritis, gangrenous dermatitis, colangiohepatitis, clostridiosis, diarrhea and/or foot pad dermatitis, in animals, preferably poultry, wherein the therapeutic composition is a compacted composition according to the present invention.

A further subject of the present invention is therefore also the treatment and/or prevention of a disease, in particular of a gut disease, preferably of necrotic enteritis, in particular of sub-clinical necrotic enteritis, in poultry, wherein at least one composition of the present invention is administered to an animal in need thereof, wherein administration includes the dissolution of the composition in water or in an aqueous solution.

A further subject of the present invention is therefore also the treatment and/or prevention of a disease, preferably a disease of poultry, selected from bacterial enteritis, gangrenous dermatitis, colangiohepatitis, clostridiosis, diarrhea and/or foot pad dermatitis, wherein at least one composition of the present invention is administered to an animal in need thereof, wherein administration includes the dissolution of the composition in water or in an aqueous solution.

The compositions of the present invention can be administered to animals in drinking water or rearing water over multiple days throughout the animal's life or during particular stages or portions of the animal's life. For example, the compositions can be administered only in a starter diet or only in a finisher diet of farm animals.

The compositions according to the invention are preferably applied to drinking or rearing water, so that the compositions are contained in the final solutions in an amount of from 10 to 500 ppm (w/w), more preferably in an amount of 20 to 400 ppm (w/w), in particular in an amount of 30 to 300 ppm (w/w) or 40 to 200 ppm (w/w) and/or in an amount, so that the spore count in the drinking or rearing water is from 1×10⁷ to 1×10¹⁰, more preferably from 1×10⁸ to 1×10⁹ CFU per liter of drinking or rearing water.

The methods of the present invention may be used for all kind of animals, in particular all kind of non-human and non-insect animals, more preferably all kind of vertebrates such as mammals, aquatic animals and birds.

Animals that may benefit from the present invention include but are not limited to farm animals, pets, exotic animals, zoo animals, aquatic animals, animals used for sports, recreation or work.

Pets are preferably selected from dogs, cats, domestic birds and domestic exotic animals. Animals used for sports are preferably horses.

Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are farming fish which are subsequently processed to give fish meal or fish oil. In this connection, the fish are preferably herring, pollack, menhaden, anchovies, capelin or cod.

In a further preferred embodiment, the animals are farm animals, which are raised for consumption or as food-producers, such as poultry, swine and ruminants.

The poultry may be selected from productive or domestic poultry, but also from fancy poultry or wild fowl. Preferred productive poultry in this context are chickens, turkeys, ducks and geese. The productive livestock in this context is preferably poultry optimized for producing young stock or poultry optimized for bearing meat. Preferred fancy poultry or wild fowl are peacocks, pheasants, partridges, chukkars, guinea fowl, quails, capercaillies, grouse, pigeons and swans, with quails being especially preferred. Further preferred poultry are ratites, in particular ostriches and emus, as well as parrots.

Ruminants according to the present invention are preferably selected from cattle, goat and sheep. In one embodiment, the compositions of this invention may be fed to preruminants to enhance their health and, in particular, to decrease the incidence of diarrhea in these animals. Preruminants are ruminants, including calves, ranging in age from birth to about twelve weeks.

Prebiotics which may be used according to the invention are preferably oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases Al (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum@ Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1, vitamin B2, vitamin B6, niacin, folic acid and panthothenate, e.g. Ca-D-panthothenate, or combinations thereof.

Immmune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof.

Amino acids which may be used according to the invention are for example lysine, alanine, threonine, methionine, valine or tryptophan, or combinations thereof.

Organic acids which may be used according to the invention are in particular C₁₋₁₀ organic acids as well as salts and esters thereof, with C₄-C₁₀ organic acids and salts and as esters thereof being preferred. Examples of such organic acids are in particular acetic acid, alpha ketoglutaric acid, arsanilic acid, benzoic acid, citric acid, ferulic acid, formic acid, fumaric acid, gallic acid, gluconic acid, humic acid, ketoisocaproic acid, lactic acid, malic acid, propionic acid, resin acid.

In case that some components are listed in different kinds of groups above or otherwise due to their characteristics are assignable to such different kinds of groups, then the preferred upper limit of the component in the composition is determined by the limit of the group having the highest upper limit and the preferred lower limit of the component in the composition is determined by the limit of the group having the lowest lower limit. Further, in such a case, the amount of the component is only taken into consideration for the preferred total amounts of the group which has the highest upper limit.

### WORKING EXAMPLES

### Example 1

### Determination of the Dissolution Time of Tablets Containing Probiotics

Powders containing differing amounts of citric acid, betaine hydrochloride, sodium bicarbonate, sodium carbonate, silica and probiotics (B. amyloliqufaciens with an average particle size (d50) of about 0.9 μm) as disclosed in table 1 were prepared by mixing the components in the amounts as disclosed in table 1. The total amount of citric acid plus betaine hydrochloride corresponds to the same total amount of proton donor in each of the prepared samples. The powders thus obtained were thoroughly mixed making use of an Eirich mixer EL 1 for 240 seconds at 20 m/sec and eventually granulated for 45 seconds at 15 m/sec.

2.3 g of the granulated compositions were subsequently pressed to a circular tablet by applying a pressure force of 30 kN, respectively.

The tablets thus obtained were subsequently dissolved in 5 L of water and the dissolution time was determined.

As can be seen from the table the dissolution time of the tablet containing only betaine hydrochloride as proton donor was the best. The higher the amount of citric acid, the lower the dissolution time. Further the increasing amount of citric acid in the compositions did also lead to increasing problems with the release of the tablets from the matrix of the pressing machine.

Further it was observed that the probiotics were homogeneously distributed in the water reservoir.

TABLE 1
Tablet compositions and dissolution time

| Component [g] | 1 | 2 | 3 | 4 | 5 | 6 |
|---|---|---|---|---|---|---|
| Citric Acid | 0.0 | 28.0 | 39.1 | 51.3 | 79.8 | 136.7 |
| Betaine HCl | 180.30 | 143.4 | 128.7 | 112.6 | 75.0 | 0 |
| NaHCO3 | 9.9 | 11.2 | 11.8 | 12.3 | 13.7 | 16.4 |
| Na2CO3 | 56.0 | 63.6 | 66.6 | 69.9 | 77.6 | 93.1 |
| Aerosil 200F | 3.8 | 3.8 | 3.8 | 3.8 | 3.8 | 3.8 |
| B. amyloliquefaciens | 27.3 | 27.3 | 27.3 | 27.3 | 27.3 | 27.3 |
| CECT 5940 | | | | | | |
| Dissolution time [s] | 76 | 88 | 90 | 96 | 101 | 105 |

### Example 2

### Qualitative Characteristics of a Tablet According to the Invention

To determine the qualitative characteristics, i.e. in particular the user-friendliness, of a tablet according to the invention in comparison to a probiotics powder with sugar-based carrier as commercially available in the market, the differences were tested by dissolving a tablet according to the invention, on the one hand, and a commercially available probiotics powder, on the other hand, in 1000 L water contained in a water tank, adjusting a probiotics concentration of 5×1011 CFU/1000 L water. The observed characteristics are summarized in the following table.

TABLE 2
Comparison of qualitative characteristics of a tablet according
to the invention in comparison to a probiotics powder with
sugar-based carrier as obtainable in the market

| | Probiotics powder | |
|---|---|---|
| | with sugar-based | Tablet according |
| Product | carrier | to the invention |
| Dosing | 5 × 10\hat{ }11 CFU/ | 5 × 10\hat{ }11 CFU/ |
| | 1000 L water | 1000 L water |
| Handling | complicated | easy |
| Dust formation | moderate | non-existent |
| Caking behaviour | strong | non-existent |
| Amount of probiotic | about 10 wt.-% | about 11 wt.-% |
| in the product | | |
| Distribution in water | Stirring is necessary | Homogeneous |
| | | distribution |
| | | without stirring |
| Sedimentation | Sedimentation takes | Homogeneous |
| behavior/distribution | place soon, if | distribution |
| of spores over time | stirring is stopped | is maintained |
| | | without stirring |
| | | for more than |
| | | 24 hours |

### Example 3

### Determination of Biofilm Formation

To determine the biofilm formation in comparison to a commercially available sugar containing probiotic, tablets were prepared, on the one hand tablets according to the invention, on the other hand tablets, where the probiotic was replaced in the preparation of the tablet by a commercially available probiotic powder with sugar-based carrier. As a negative control, a tablet without probiotics was prepared.

The tablets according to the invention contained the following components: 11 wt.-% probiotics (B. amyloliquefaciens CECT 5940, 51 wt.-% betaine HCl, 10 wt.-% citric acid, 23 wt.-% sodium carbonate, 4 wt.-% sodium hydrogen carbonate, 1.5% fumed silica (Aerosil® 200F). As comparative example tablets with the same ingredients were prepared where the probiotic was replaced by a commercially available sugar containing probiotics powder.

To simulate the conditions of drinking water lines as employed in chicken farming, the different formulations were dissolved in water (process water, 20° C.) to adjust a probiotics concentration in the water of about 5×10\hat{ } 8 CFU/L. The probiotics containing water was then pumped through a system consisting of 3 Schott 1L flasks, which were connected by PVC and stainless steel tubes with a diameter of 4 mm and a length of 300 mm, respectively. The water in the Schott flasks was continuously stirred. Pumping of the water was established by using 3 Watson Marlow 120U pumps. The probiotics containing solutions were replaced daily. Pumping took place 16 h each day, simulating the conditions of poultry farming, where the poultry rests for about 8 hours each day. The flow velocity was increased from 9.8 mL/min in the first week up to 46.2 mL/min in week 6.

After 6 weeks the amount of biofilm formation was determined by using crystal violet for staining the biofilm as formed in the PVC and stainless steel tubes. Staining of the biofilm was carried out as described by George A. O'Toole (2011), Microtiter Dish Biofilm Formation Assay. JoVE. 47. http://www.jove.com/details.php?id=2437, doi: 10.3791/2437. The more biofilm was formed, the stronger is the staining. The strength of staining was determined by measuring the optical density at a wavelength of 550 nm (OD₅₅₀). The results are presented in the following table.

TABLE 3
Biofilm determination by crystal violet staining

| | Negativ | Tablets of the | Tablets with probiotic |
|---|---|---|---|
| Material | control | invention | on sugar-based carrier |
| PVC tubes | 2.84 | 3.19 | 17.3 |
| Stainless steel tubes | 2.62 | 2.60 | 12.4 |

The experimental data show that by using tablets of the invention, the biofilm formation can be minimized. The biofilm formation is almost the same as for the negative control, i.e. like by using a tablet which does not contain probiotics, at all. Replacing the probiotic in the tablet by a commercially available sugar containing probiotic, to the contrary, leads to a significant undesired increase of the biofilm formation.

### Example 4

### Feeding Trials

The farm consisted of 6 houses with about 35.000 birds per house. The poultry used in the trial were Ross 308, mixed sex, with an average start weight of 40 g. The poultry was fed with a standard diet for 40 days. Three houses (treatment group) were supplied with probiotic B. amyloliquefaciens CECT 59 contained in the drinking water, whereas three other houses (control group) were not supplied with probiotic.

Supply with probiotic of the treatment group was carried out as follows:

An 8 g tablet with about 4.5 Ell CFU/tablet was dissolved in 20 L of water to prepare a stock solution. The tablet contained 0,8 g citric acid, 4.0 g betaine hydrochloride, 0.3 g sodium hydrogen carbonate, 1.8 g sodium carbonate, 0.9 g B. amyloliquefaciens CECT 59 (Ecobiol®), 0.1 g fumed silica (Aerosil® 200F).

The stock solution was supplied through the drinking lines at a ratio of 1:50 to the poultry for 12 hours on each of days 0-2, 10-12, 21-23 and 27-29.

The results of the feeding trial are summarized in the following table.

TABLE 4
Effect of probiotic supply on feeding parameters

| Parameter | Farm average | Control group | Treatment group |
|---|---|---|---|
| EPFE | 414.27 | 414.49 | 141.04 |
| Av BWG | 2.247 | 2.230 | 2.264 |
| Hock % | 27.15 | 29.72 | 24.58 |
| Podo % | 22.56 | 25.30 | 19.82 |
| Factory rejects | 0.43 | 0.49 | 0.38 |

The application of the probiotic containing tablets to the drinking water resulted in an increase of the average body weight of the poultry of in average 34 g per bird over the three houses. The application further led to a reduction of hock burns and pododermatitis of more than 5% in the treated poultry in comparison to the untreated poultry. Furthermore, the number of factory rejects was significantly reduced.

1. A composition comprising at least one betaine salt and at least one feed additive.

2. The composition according to claim 1, wherein the composition contains the at least one betaine salt in an amount of 20 to 90 wt. %.

3. The composition according to claim 1, wherein the composition contains at least one carbonate salt.

4. The composition according to claim 1, wherein the composition contains at least one bicarbonate salt.

5. Composition The composition according to claim 1, wherein the composition contains at least one polyfunctional acid.

6. The composition according to claim 1, wherein the composition contains at least one further compound selected from the group consisting of disintegrants, flow regulators, anti-caking agents, lubricants, emulsifiers, antioxidants, bulking agents, gelatinizers, colouring agents, and flavouring agents.

7. The composition, according to claim 1, wherein the at least one feed additive is selected from the group consisting of probiotics, minerals, vitamins, enzymes, probiotics, amino acids, organic acids, vaccines, immune modulators, and mixtures thereof.

8. The composition according to claim 1, wherein the composition contains at least one probiotic microorganism.

9. The composition according to claim 8, wherein the at least one probiotic microorganism is selected from the group consisting of probiotic bacteria.

10. The composition according to claim 1, wherein the composition is a compacted composition.

11. A method of preparing a compacted composition, the method comprising:
a) providing the composition according to claim 1, wherein the composition is in the form of a powder;
b) granulating the powder to prepare a granular composition; and
c) pressing the granular composition to form the compacted composition.

12. The method according to claim 11, wherein pressing of the granular composition is carried out by applying a pressure force of 5 to 100 kN.

13. A method of providing a feed additive animals, the method comprising:
a) dissolving the composition according to claim 1 in water or an aqueous solution, to obtain a prepared solution; and
b) providing the prepared solution to animals.

14. The method according to claim 13, wherein the animals are selected from the group consisting of farm animals, pets, exotic animals, zoo animals, aquatic animals, and animals used for sports, recreation, or work.

15. The method according to claim 13, wherein provision of the feed additive to the animals enhances the health of such animals, and/or improves the general physical condition of such animals, and/or improves the feed conversion rate of such animals, and/or decreases the mortality rate of such animals, and/or increases the survival rates of such animals, and/or improves the weight gain of such animals, and/or increases the productivity of such animals, and/or increases the disease resistance of such animals, and/or increases the immune response of such animals, and/or establishes or maintains a healthy gut microflora in such animals, and/or prevents the outgrowth of pathogenic bacteria, and/or enhances the growth of beneficial bacteria, and; or reduces the pathogen shedding through the feces of such animals.

16. The composition according to claim 2, wherein the composition contains the at least one betaine salt in an amount of 45 to 65 wt.-%, and
wherein the at least one betaine salt is betaine hydrochloride.

17. The composition according to claim 3, wherein the composition contains the at least one carbonate salt in an amount of 10 to 35 wt.-%, and
wherein the at least one carbonate salt is sodium carbonate.

18. The composition according to claim 4, wherein the composition contains the at least one bicarbonate salt in an amount of 1 to 10 wt.-%, and
wherein the at least one bicarbonate salt is sodium bicarbonate.

19. The composition according to claim 9, wherein the at least one probiotic microorganism is selected from the group consisting of probiotic bacteria from B. subtilis, B. iichenybrinis, B. amylolyquiefaciens, E. faecium, and mixtures thereof.

20. The composition according to claim 10, wherein the compacted composition is in a shape of a tablet.
